(19) United States
(12) Patent Application Publication
(10) Pub. No.: US 2025/0255473 A1
(43) Pub. Date: Aug. 14, 2025

(54) DEVICES AND METHODS FOR TWO CHANNEL BRONCHOSCOPY

(71) Applicant: Beth Israel Deaconess Medical Center, Inc., Boston, MA (US)

(72) Inventors: Fayez Kheir, Boston, MA (US); Adnan Majid, Boston, MA (US); Daniel Ospina-Delgado, Boston, MA (US)

(21) Appl. No.: 19/049,876

(22) Filed: Feb. 10, 2025

Related U.S. Application Data
(60) Provisional application No. 63/551,642, filed on Feb. 9, 2024.

(51) Int. Cl. A61B 1/267; A61B 1/00; A61B 1/005; A61B 1/015; A61B 1/018; A61B 1/04; A61B 1/07; A61B 17/00

(52) U.S. Cl. CPC A61B 1/2676; A61B 1/00105; A61B 1/00128; A61B 1/0051; A61B 1/015; A61B 1/018; A61B 1/04; A61B 1/07; A61B 17/00234; A61B 2017/003; A61B 2017/0034; A61B 2217/005

(57) ABSTRACT

The present disclosure relates to bronchoscope devices and method of use for the visualization and surgical treatment of pulmonary conditions within the lungs of a patient. The bronchoscope assembly comprises a first working channel and a second working channel that respectively provide complementary functions for accessing the surgical site and a plurality of surgical applications.

This application claims priority to U.S. Provisional Application No. 63/551,642 filed on Feb. 9, 2024. The entire contents of the above application is incorporated herein by reference.

### BACKGROUND

Bronchoscopes have been developed for viewing of the lungs to aid in the diagnosis and disease. Many such devices have been provided with a flexible tip to aid in directional navigation of the bronchoscope into the bronchial tree of the lungs so that conditions can be visualized that impact respiratory function. It is often desirable to perform further procedures such as the biopsy of tissue samples from the bronchial system to aid in diagnosis, the delivery and removal of fluid that can impact oxygenation by the lungs and the delivery of supplemental oxygen should this essential function of the lungs be impaired.

Existing endoscopes typically include a camera to image tissue, a light source for illuminating the lungs during bronchoscopy and a working channel by which various functions can be performed during such procedures. As the airway passages within the bronchial tree get progressively smaller as the tip of the bronchoscope is steered from the trachea into smaller passageways so that the distal diameter of the bronchoscope must be limited. Thus, there is limited cross-sectional area at the distal end of the endoscope insertion tube with the working channel being 2 mm in diameter or less, for example, so that the remainder of the distal surface area can also accommodate the imaging and illumination apertures needed for visualization. These apertures typically use a light guide lens for illumination and an objective lens to couple light from the field of view to the imaging device. Flexible bronchoscopes must include a mechanical mechanism to adjust the angular orientation of the distal end relative to the longitudinal axis of the insertion tube that extends through the trachea. This typically entails the use of wires that extend through the insertion tube wall to apply tension on different sides of the distal end of the bronchoscope so that the user can manipulate a handle mechanism to steer the distal end as required. This typically requires a wide angulation range of over 100 degrees in two opposite directions to achieve to achieve the desired steerability.

As the working channel must be used to provide these various different functions, time is needed to transition between the different procedures. If a biopsy is being performed, for example, this requires a forceps tool to be inserted through the working channel, which thus prevents the working channel from serving other functions until the forceps has been removed. If fluid in the lungs impairs viewing during the biopsy, it can be necessary to remove the biopsy tool, attach a source for suctioning of the fluid to the working channel, remove the fluid to restore adequate imaging of the biopsy site, remove the suction and return the biopsy tool through the working channel. Consequently, procedures can extend for many hours to sequence through the various steps needed to complete the procedure. In the case of an hypoxic patient, it can be necessary to supply supplemental oxygen to the patient through the working channel periodically during the procedure or risk the occurrence of hypoxia which can affect the patient for many hours after completion of the procedure. Biopsies are conducted using a flexible bronchoscope as described herein where a biopsy probe can be inserted into the lung and related anatomical structures described and illustrated herein to remove tissue and/or fluid samples from different location within the bronchial tree including lymph nodes that can be accessed using the working channels described herein.

Thus, a need exists for further improvements in bronchoscope design and function to improve the treatment of patients with pulmonary disease or injury.

### SUMMARY

Preferred implementations of the invention provide for the use of a second working channel for bronchoscopy procedures that enable the insertion of the flexible bronchoscope into the bronchial tree for visualization and treatment. This eliminates the need to disrupt a procedure where a second working channel can be used to provide additional operative functions while a first working channel is being used for a critical procedural function.

A preferred embodiment utilizes an adapter that can be attached to an existing bronchoscope in which the adapter includes a working channel that works separately from the existing bronchoscope working channel. The adapter thus comprises a second working channel that can be used in tandem with the first working channel of the existing bronchoscope. The adapter can include a proximal assembly, a tube attached to the exterior surface of the bronchoscope or a shaped body that conforms to the exterior surface wherein the tube or shaped body extends from the proximal assembly to a flexible distal end having a distal aperture for the second working channel.

The adapter can have a plurality of operative elements including a second working channel port, a connector that connects to a negative pressure source to provide suction and a suction actuator to facilitate the application of suction to the working channel. The port can include a y connector to sidewall access to the adapter working channel.

The adapter working channel can comprise a plurality of different shapes and sizes to enable the user to utilize the adapter working channel for different applications. The adapter can consequently be configured to be use the additional working channel for the hypoxic patient that requires a flexible bronchoscopy. It has been reported that up to 35% of patients that undergo bronchoscopy and do not receive any kind of oxygen supplementation can develop hypoxemia (See Milman N, Faurschou P, Grode G, Jørgensen A. Pulse oximetry during fibreoptic bronchoscopy in local anaesthesia: Frequency of hypoxaemia and effect of oxygen supplementation. Respiration. 1994; 61(6):342-347. doi:10.1159/000196366). Patients diagnosed with COPD, asthma, COVID, OSA, or other diseases that impair the gas exchange in the lungs or oxygen delivery might develop hypoxia during the procedure that can extend up to 4 hours postoperatively. This limits the ability of the bronchoscopist to navigate the airways, prolongs the surgical time, and can lead to situations in which it is hard to recover adequate oxygen saturation or even to hypercapnic respiratory failure. In their guidelines for diagnostic flexible bronchoscopy, the British Thoracic Society (See Du Rand I A, Blaikley J, Booton R, et al. British Thoracic Society guideline for diagnostic flexible bronchoscopy in adults. Thorax. 2013; 68(SUPPL. 1). doi:10.1136/thoraxjnl-2013-203618) recommends using oxygen supplementation when desaturation is significant (pulse oximeter oxygen saturation (SpO2)>4% change, or SpO2<90%) and prolonged (>1 min) to reduce the risk of hypoxemia-related complications. This can prove challenging in most occasions in which the working channel of the bronchoscope is used, as patients can only be ventilated through the area in between the inner diameter of the endotracheal tube and the outer diameter of the bronchoscope, unless the bronchoscope is removed.

Tracheal oxygen supplementation through a working channel has previously proven to be feasible and safe in preventing and correcting hypoxemia during flexible bronchoscopy, with the caveat of having to stop the procedure to supply the oxygen. The addition of a second working channel allows the physician to use an independent channel to supply oxygen to the patient throughout the procedure, thereby avoiding the development of hypoxia, and with it, preventable complications. The mechanism through which the tracheal supplementation of oxygen works might be similar to the one in transtracheal oxygen therapy, the anatomical dead space is bypassed with the working channel leading to better oxygen delivery and requiring less volume than when a nasal cannula is used.

Other therapeutic applications of the second working channel attachment include, but are not limited to the following procedures. With improved suction hemoptysis can be performed to provide improved cleanup before coagulation or better visualization of the lesion while ablation. Enhanced suction can address mucus plugging where concomitant irrigation helps remove thick mucus. Two tools can be used through the two working channels to facilitate manipulation and retrieval of stents and can avoid the need to redeploy. Two tools can also be used for the resection of endobronchial tumors thereby leading to better outcomes and shorter surgical times. Two tools can be used for removal of foreign bodies that are difficult to grasp.

Further applications can include whole lung lavage where one channel can be used for suction while the other can be used for irrigation, creating a loop that can greatly improves efficiency and reduces surgical times.

Additional therapeutic procedures can include bronchial thermoplasty/Rheox for patients with asthma and chronic bronchitis. Another application involves fibrin glue application for BPF/APF to prevent the two components of the fibrin glue from combining inside the working channel, rendering the bronchoscope useless. Thus, one component is delivered with a first tube through the first channel and the second component is delivered through a second tube with the second channel to allow a more precise application while combining the components at the site of glue application. Percutaneous endoscopic gastrostomy (PEG) can involve placement of a tube with a bronchoscope which can aid in insufflation of the stomach and guidewire advancement to the stomach (see Folch E, Kheir F, Mahajan A, Alape D, Ibrahim O, Shostak E, Majid A. Bronchoscope-Guided Percutaneous Endoscopic Gastrostomy Tube Placement by Interventional Pulmonologists: A Feasibility and Safety Study. J Intensive Care Med. 2020 September; 35(9):851-857. doi:10.1177/0885066618800275. Epub 2018 Sep. 24. PMID: 30244635).

Additionally, diagnostic procedures that can be enhanced with a second working channel such as cryobiopsy where suction is used while performing the biopsy to improve visualization. This enables the use of a thermoablative tool immediately after the biopsy if bleeding is detected. The biopsy sample can be grasped with another tool to avoid losing it when the probe thaws. Biopsies can be used to obtain tissue or fluid samples within the lung thoracic wall as described herein by appropriate positioning of the distal end of the bronchoscope system as described herein with a smaller diameter distal end or the insertion of a smaller diameter tool that can extend from the distal end of the bronoscope through one of the working channels described herein. A further example of a bronchoscope as described herein can comprise a single outer tube with two separate workings channels of the same or different sizes.

Another example is transbronchial biopsy where suction is used in case of bleeding to maintain a clean field of view. This further enables the use of a thermoablative tool or the placement of a tamponade balloon immediately after the biopsy if bleeding is detected. Thus the second channel can generally be used to maintain clean field of view during therapeutic bronchoscopy depending on the application.

### DESCRIPTION OF THE DRAWINGS

FIG. 1 is a perspective view of an adapter proximal assembly in accordance with preferred embodiments hereof;

FIG. 2A is an exploded view of the adapter proximal assembly of FIG. 1;

FIG. 2B is a detailed view of a suction actuator for a preferred embodiment hereof;

FIGS. 3A and 3B are perspective and end views of the distal end of a bronchoscope in connection with preferred embodiments;

FIGS. 4A and 4B are perspective and end views of a bronchoscope in connection with further preferred embodiments;

FIG. 5 is a side view of a proximal adapter assembly mounted onto an available bronchoscope;

FIG. 6 is a process flow diagram illustrating surgical procedures for preferred embodiments of the invention.

FIG. 7 illustrates the lung with the bronchial tree wherein the bronchoscope assembly described herein can be inserted and steered into smaller branches to perform a plurality of therapeutic and diagnostic procedures.

FIG. 8 illustrates a bronchoscope assembly inserted into the bronchial tree to perform administration of transtracheal oxygen.

FIGS. 9A, 9B, and 9C illustrate a bronchoscope assembly inserted into the bronchial tree to perform hemoptysis treatment.

FIGS. 10A and 10B illustrate a bronchoscope assembly inserted into the bronchial tree to perform removal of a mucus plug.

FIGS. 11A and 11B illustrate a bronchoscope assembly inserted into the bronchial tree to perform deployment, revision, or removal of a stent.

FIGS. 12A, 12B, and 12C illustrate a bronchoscope assembly inserted into the bronchial tree to perform resection of endobronchial tumors.

FIGS. 13A and 13B illustrate a bronchoscope assembly inserted into the bronchial tree to perform removal of a foreign body.

FIG. 14 schematically illustrates a bronchoscope having two working channels in a cylindrical perspective end view in which a catheter is extended distally from a working channel to access smaller bronchial channels in accordance with the implementations described herein.

### DETAILED DESCRIPTION

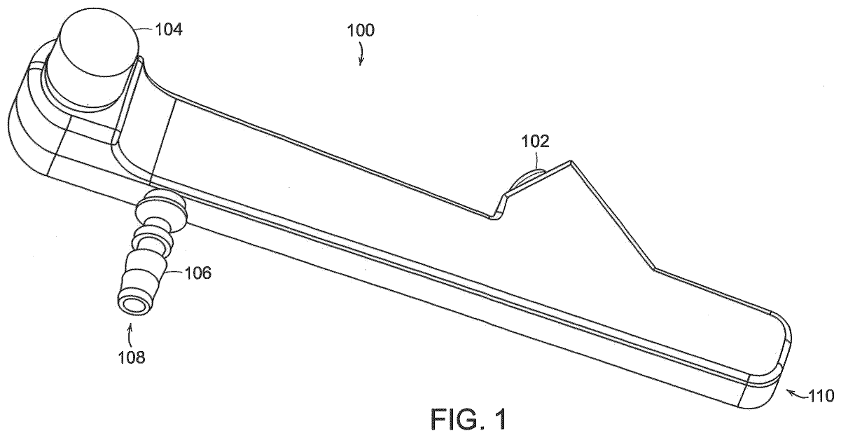

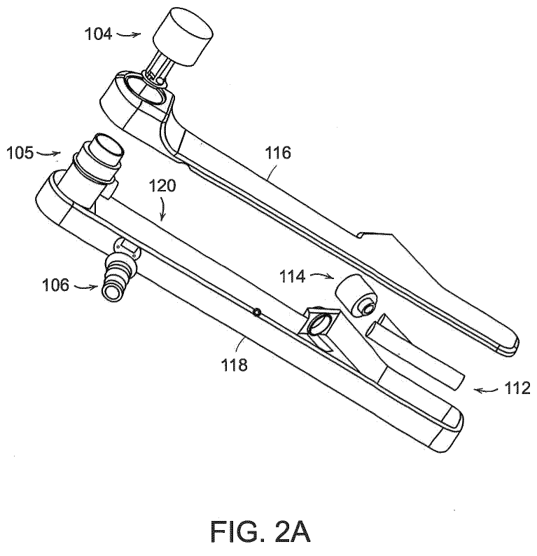

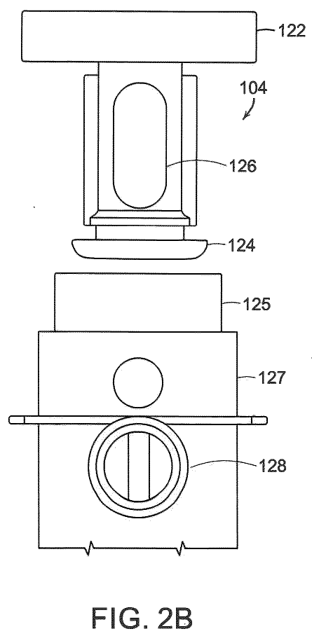

Shown in FIG. 1 is a proximal assembly of an adapter 100 for a bronchoscope in which the proximal assembly comprises a housing 110 that can be formed of two molded components that can be joined and configured to attach to a bronchoscope. The proximal assembly housing 110 can include a port 102 which can be located on a first side of housing 110 so that instruments can be inserted into an internal working channel that extends into a tube attached to a distal end of the proximal assembly. At the proximal end of the housing 110 a valve 104 can be positioned that enables a user to quickly actuate fluid flow within the adapter working channel. The valve 104 can have a button or knob that can be manually adjusted by the user to open, close, or regulate fluid flow within the working channel. A second port 106 on a second side of the housing can be used to attach a suction tube or other fluid source to the housing thereby enabling fluid flow into or out of the proximal assembly. Note that fluid can also be introduced through port 102. As seen in the exploded view of FIG. 2A, the housing 110 can include a working channel chamber 114 coupled to the port 102 aperture where the chamber 114 is connected at one end to one arm of a y connector 112 at port 102 so as to provide entry into the working channel exiting the distal end of housing 110. The second arm of connector 112 is in fluid communication with the valve chamber 105 which is in fluid communication with second port connector 106 so that fluid can flow into or out of aperture 108 of port 106 so as to enable fluid flow between aperture 108 and the adapter working channel.

As seen in the exploded view of valve 104 in FIG. 2B, the valve can include a manually grasped knob or button used to operate the valve, an internal valve body 104 with an opening 126 such that the valve chamber 105 can be moved or rotated to align opening 126 with the working channel port 128 in the external valve body 127 that is statically connected to lower section 118 of housing 110. In this exemplary implementation, knob 122 is rotated relative to upper section 116 of housing 110. The internal valve body 104 is placed within the external valve body 127 through an opening in upper rim 125 that has an inner cylindrical wall contacted by seal 124 that forms a fluid tight seal with the inner cylindrical wall.

The distal end of the proximal assembly is connected to an outer wall 107 of the adapter working channel. The outer wall 107 can comprise a tube or shaped housing that attaches to an exterior surface of the bronchoscope insertion tube 22. As seen in the exemplary implementation of FIGS. 3A and 3B, the outer wall 107 can have a concave surface that attaches to the outer convex surface of insertion tube 22. In this implementation a flange 144 can be snap connected to the exterior surface of tube 22, although various mechanical means can be used to provide for attachment of the outer wall 107 of the adapter and the surface of tube 22. In this embodiment, the bronchoscope has a distal imaging channel 146, a distal illumination channel 148, and a distal aperture for the bronchoscope working channel. The adapter working channel 145 has an outer wall 142 wherein the aperture can have a non-circular shape to maximize the channel area while keeping the width W of the adapter working channel 145 smaller than the diameter R of the bronchoscope working channel 150.

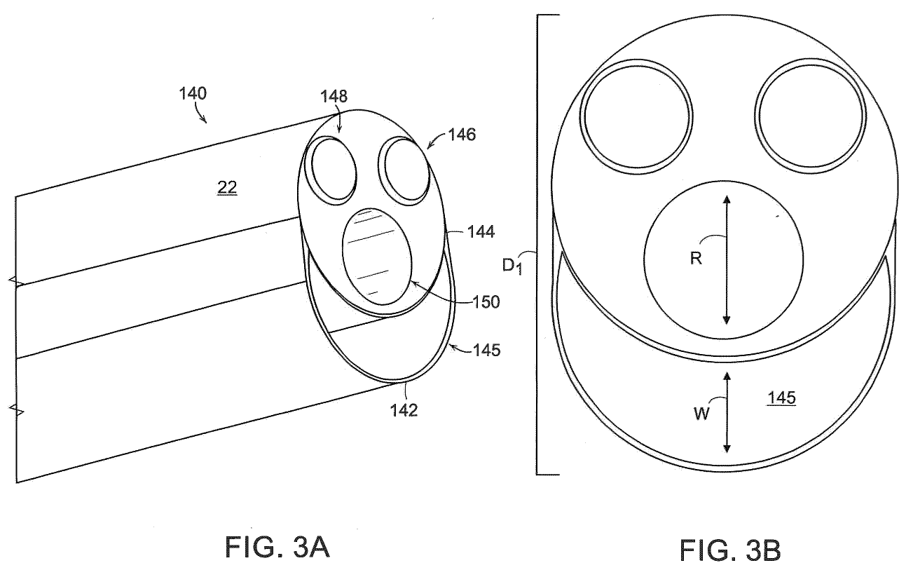

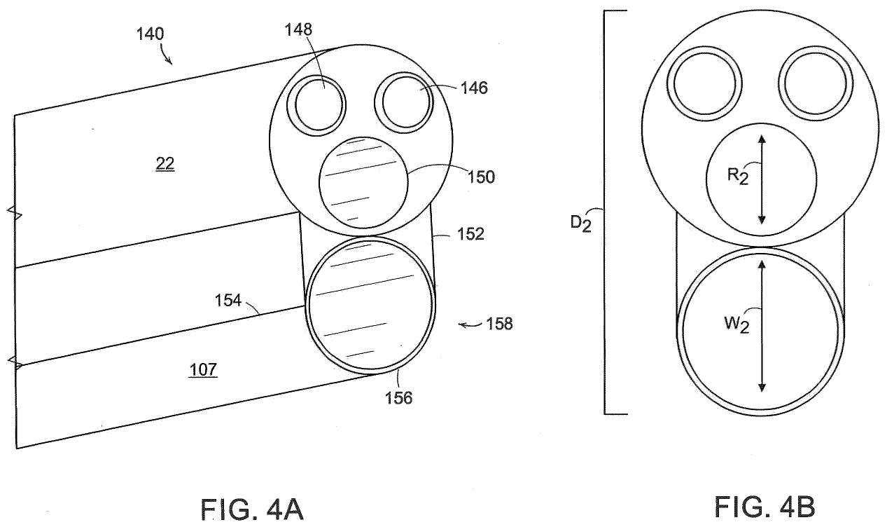

In the exemplary embodiment of FIGS. 4A and 4B, the bronchoscope can have similar features to the bronchoscope of FIGS. 3A and 3B, for example, or the features can have different dimensions and operative characteristics, however, the adapter working channel in this embodiment has a circular geometry with a diameter W2 that is either larger (as shown) than the diameter R2 of the bronchoscope working channel, or alternatively can be the same size, or smaller, depending the requirements of the procedure. Note that the diameter DI along the long axis of the assembly in FIG. 3B can be greater or less than the diameter D2 along the long axis of FIG. 4B. The diameters D1 and D2 can be selected depending upon the order (or size) of the bronchi in which the surgical procedure is to be performed within the bronchial tree of the patient. The smaller bronchi further into the bronchial tree require a smaller diameter for the assembly. As bronchoscope insertion tubes can vary in size from about 4 mm in diameter (less than 3 mm in pediatric patients) to about 9 mm in diameter, and working channel sizes can vary depending upon the application and needs for a particular procedure. The overall diameters D₁ and D₂ can be in the range of 3 mm to 6 mm, or alternatively, in the range of 4 mm to about 7 mm to provide the desired two working channel flexible bronchoscope. Further embodiments can comprise a single cylindrical bronchoscope tube for insertion into the lungs having two working channels. The two working channels can be the same size, or have different sizes as described herein to facilitate the use of two different tools depending on the specific application. The single bronchoscope implementation is preferably less than 6 mm in diameter, but can be larger for certain applications such as when a small diameter catheter can be used through one of the working channels to reach smaller bronchial features within the bronchial tree such as the alveoli.

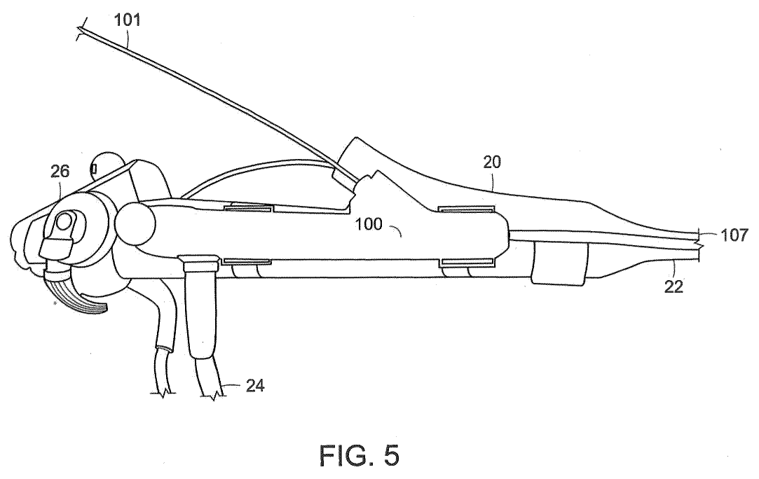

Shown in FIG. 5 is an exemplary adapter 100 mounted onto a bronchoscope handle assembly 20 in which the bronchoscope handle has an actuator 26 to operate the bronchoscope tip angulation for navigating the tip through different stages of the bronchial tree. The bronchoscope handle 20 can include an imaging sensor and/or light source 26 at the proximal end that is coupled to the distal end with a plurality of optical fibers configured as an imaging bundle 146, and/or illumination bundle or optical fiber array 148, respectively, at the distal end. Alternatively, the imaging sensor can be a distally mounted small diameter imaging chip such as those available from Omnivision Technologies, Santa Clara, California, such as the OV6948, which has an outer diameter of less than 1 mm and a resolution or 400 pixels with an integrated distal lens. Such distal imaging detectors at 146 can have diameters of 3 mm or less (e.g. OVM6946) with 400×400 pixels and including an onboard controller and serial peripheral interface (SPI) that outputs 30 frames per second for video output. The insertion tube 107 of the adapter in which the adapter working channel extends can be statically mounted to the outer wall surface of the bronchoscope insertion tube 22. The static mounting of the handle assembly can optionally use flaps that that grasp the bronchoscope handle, that rigidly attach to the bronchoscope handle with elastic bands with retention elements that engage the bronchoscope handle and/or that use Velcro, tape, adhesive and or double-sided microsuction tape to adhere to surfaces on the bronchoscope handle. A tool or fluid insertion tube 101 is shown inserted into the adapter port 102. A suction tube 24 can be attached to the port 106 of the adapter housing 110.

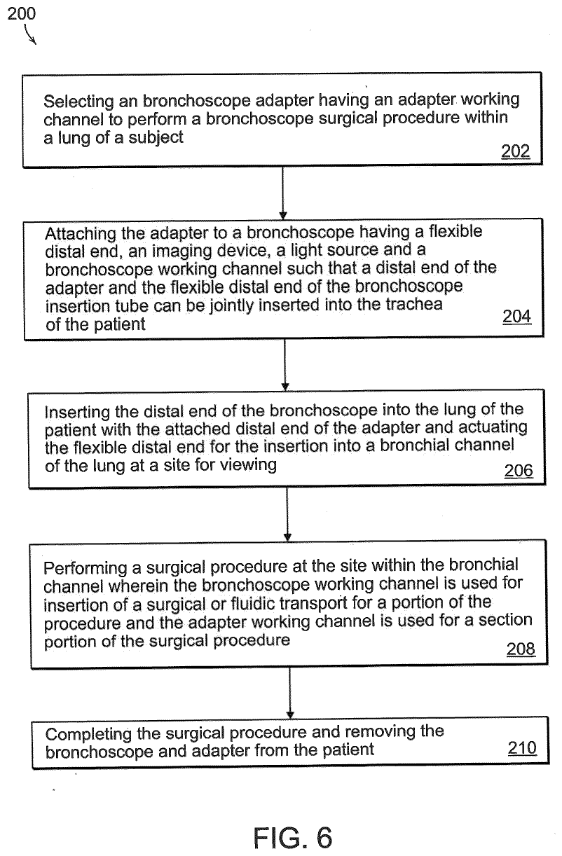

Shown in FIG. 6 is an exemplary surgical procedure illustrating a method 200 of using the adapter with a bronchoscope as generally described herein. The user, such as a bronchoscopic surgeon, selects a bronchoscope having a diameter and operating characteristics suitable for the procedure and then selects 202 an adapter configured to be mounted on the selected bronchoscope. The adapter is attached 204 to the bronchoscope which typically has a steerable distal end having the required flexibility at the distal end of the insertion tube for insertion into the bronchial tree of the patient. The flexibility of the distal end of the adapter is configured to match the distal flexibility of the bronchoscope. Similar biocompatible materials are used in both components to enable complementary movement with a smooth surface for tissue contact along the outer surfaces.

Optionally, an outer sheath, such as an elastic outer sleeve, can be placed over both components at the distal end to stabilize the distal movement of the assembly and provide a smooth frictionless tissue contact surface. After insertion 206 through the trachea and into a lung of the patient, the distal end is steered into the bronchial tree for visualization of the surgical site. The procedure is performed 208 wherein a certain portion of the procedure is conducted through the bronchoscope working channel and a second portion of the procedure is performed through the adaptor working channel. After completing the surgical procedure, the bronchoscope and adapter are removed 210 from the patient. The distal imaging of the procedure can be recorded and stored in the patient medical record. The adapter can comprise a single use disposable product or can be partially or completely sterilized after use. For example, the insertion portion of the adapter can be removed and disposed whereas the adapter assembly can be fabricated with materials for sterilization.

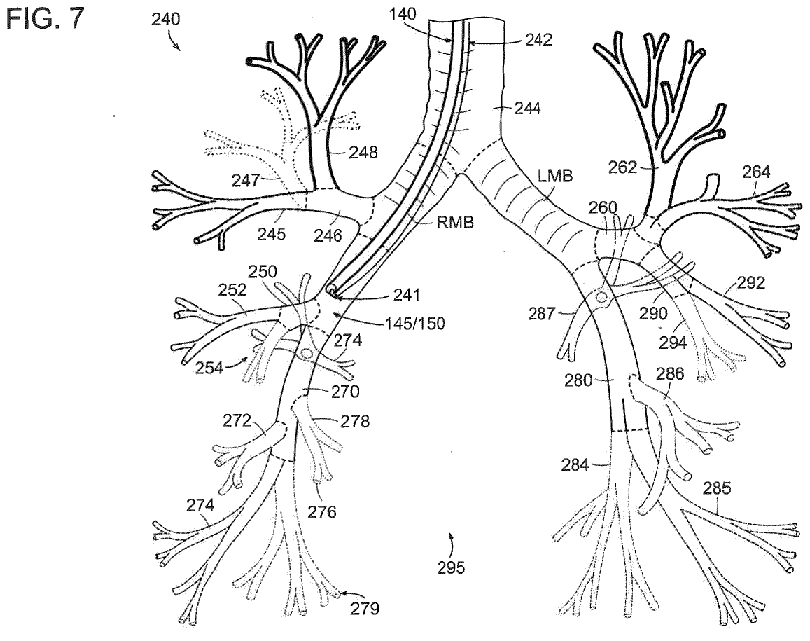

Illustrated in FIG. 7 is a sectional view of a lung 240 that is bounded by the thoracic wall surrounding the pleural cavity that includes the bronchial tree. The bronchial tree includes the right upper lobe 246 with the anterior 245, posterior 247 and apical 248 segments, the right middle lobe 250 with the lateral 252 and medial 254 segments, the right lower lobe 270 with the superior 271 lateral 272, posterior 274, anterior 278, and medial 278 segments, the left upper lobe 260 with the apico-posterior 262 and anterior 264 segments, the linguia 290 with the superior 292 and inferior 294 segments, and the left lower lobe 280 with the anteromedial 284, superior 285, posterior 286 and lateral 287 segments. The ends of the lobes extend to alveoli 279 that transport oxygen into the blood. The scope 140 as described herein has a distal end that can be steered into smaller branches of the system so that the distal openings of working channels 145 and/or 150 can be positioned adjacent to tissue to be treated whereby a tool can be inserted through one of the working channels such a distal end 241 of a tool, such as a biopsy tool can be positioned within a larger or smaller branch for treatment.

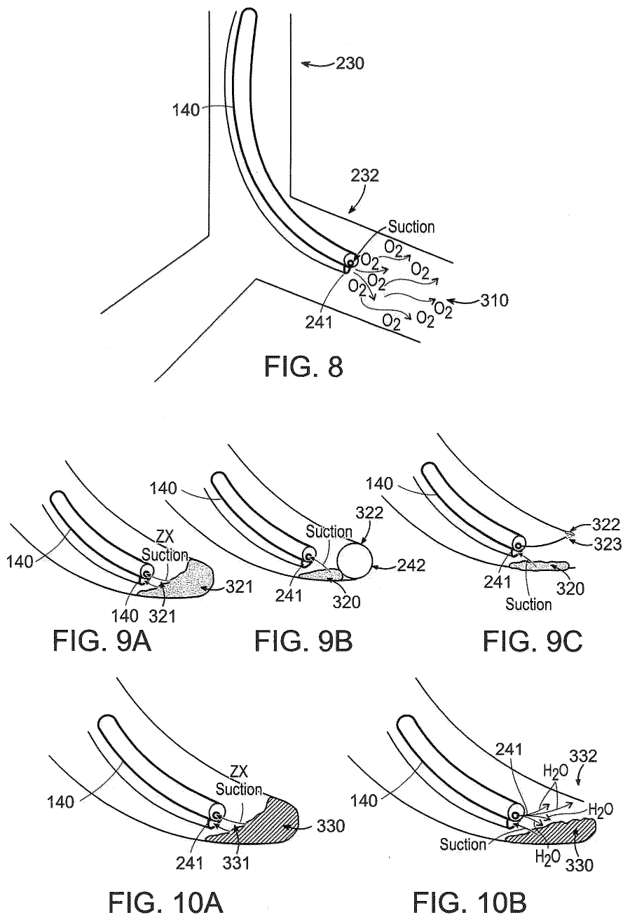

The small tools can have diameters less than 4 mm, less than 3 mm and less than 2 mm depending on their function and the depth within the bronchial tree required for placement. The tools can include biopsy forceps, cytology brushes, transbronchial needle aspiration tools (TBNA needles, a grasping tool to provide for removal of foreign bodies within the lungs, coagulation electrodes, a spatula knife or other cutting tool, a spray catheter, a balloon catheter, an airway sizing kit for bronchial valves and the delivery of such valves, and a tool or catheter for stent delivery. Exemplary tools and use thereof are described with regards to FIGS. 8-13B.

FIG. 8 illustrates a bronchoscope assembly 140 inserted into the bronchial tree to perform administration of transtracheal oxygen 310. The assembly 140 having two working channels is useful for treating a hypoxic patient. Up to 35% of patients who undergo bronchoscopy and do not receive any kind of oxygen supplementation can develop hypoxemia. Patients diagnosed with COPD, asthma, COVID, OSA, or other diseases that impair the gas exchange in the lungs or oxygen delivery are at a high risk of developing hypoxia during the procedure, which can linger up to four hours postoperatively. This limits the ability of bronchoscopist to navigate the airways, prolongs the surgical times, and can lead to situations in which it is hard to recover adequate oxygen saturation or even hypercapnic respiratory failure. In their guidelines for diagnostic flexible bronchoscopy, the British Thoracic Society recommends using oxygen supplementation when desaturation is significant (pulse oximeter oxygen saturation (SpO₂)>4% change or SpO₂<90% and prolonged (>1 min) to reduce risk of hypoxemia-related complications. When the working channel of a conventional bronchoscope is being used, the patient can only be ventilated through the area between the inner diameter of the endotracheal tube and the outer diameter of the bronchoscope unless the bronchoscope is removed, leading to a higher risk of hypoxia.

Tracheal oxygen supplementation through a single working channel has previously proven to be feasible and safe in preventing and correcting hypoxemia during flexible bronchoscopy, with the caveat of having to stop the procedure to supply oxygen. The addition of a second working channel allows the physician to use an independent channel to supply oxygen to the patient throughout the procedure, avoiding the development of hypoxia and with it preventable complications. The mechanism through which the tracheal supplementation of oxygen works might be similar to the one in transtracheal oxygen therapy; the anatomical dead space is bypassed with the working channel leading to a better oxygen delivery to respiratory bronchioles and alveoli with a requirement of fewer litters than when a nasal cannula is used.

FIGS. 9A, 9B, and 9C illustrate a bronchoscope assembly 140 inserted into the bronchial tree to perform hemoptysis treatment. The second working channel of the assembly 140 enhances suction 321 and enables the use of an occlusion balloon 322 to stop bleeding and better locate the lesion 320 in the bronchial tree while maintaining adequate suction through the bronchoscope assembly's 140 main working channel. Furthermore, once the lesion 320 is identified, the second working channel allows for continued use of the main working channel, providing improved suction that enhances cleanup before coagulation occurs and offers better visualization of the lesion 320 while an ablation tool 323 is utilized via the second working channel, potentially reducing surgical time and complications.

FIGS. 10A and 10B illustrate a bronchoscope assembly 140 inserted into the bronchial tree to perform removal of a mucus plug 330. In this embodiment, the second working channel can serve as a secondary suction port 331 to improve the removal of the mucus plug 330 or to simultaneously irrigate 332 the airway while suctioning, which could soften the mucus plug 330 and help eliminate thick mucus in the airways.

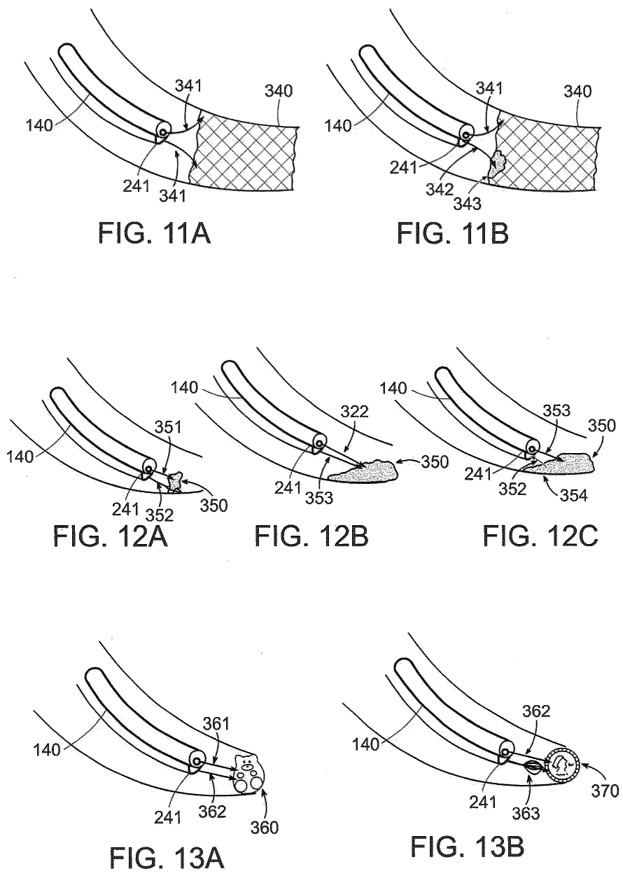

FIGS. 11A and 11B illustrate a bronchoscope assembly 140 inserted into the bronchial tree to perform deployment, revision, or removal of a stent 340. The second working channel may allow for the simultaneous use of two forceps 341 during stent deployment, which can facilitate the manipulation of tracheal and bronchial stents, avoiding the need to redeploy the stent 340 and thereby reducing surgical times and potential medical waste. The second working channel may allow the bronchoscopist to use a second forceps 341 or an ablation device 342 to better handle the stent and remove granulation tissue 343 when necessary.

FIGS. 12A, 12B, and 12C illustrate a bronchoscope assembly 140 inserted into the bronchial tree to perform resection of endobronchial tumors 350. The second working channel may allow for use of two tools, for example a forceps 351 and an ablation catheter 352, a forceps 351 and a snare 353, a forceps 351 and a balloon, ablation, and suction 353, and many other combinations that may lead to better surgical outcomes and shorter surgical times while providing more flexibility to the bronchoscopist performing the procedure.

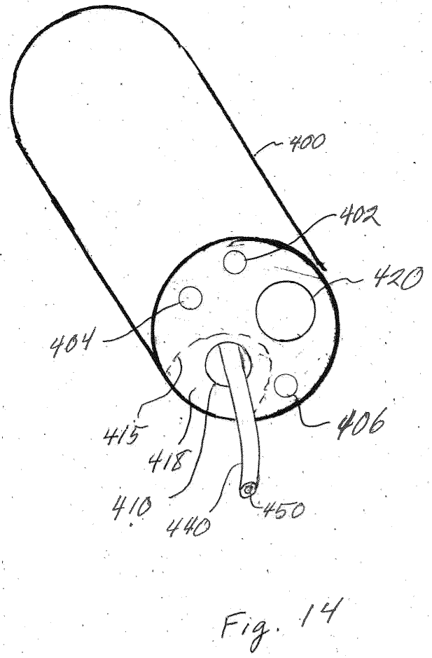

FIGS. 13A and 13B illustrate a bronchoscope assembly 140 inserted into the bronchial tree to perform removal of a foreign body 360. The second working channel may enable bronchoscopists to simultaneously use a basket 363 with forceps 362 or use forceps 362 with a cryoprobe 361 to more effectively grasp foreign bodies that might otherwise be difficult to handle or could come loose and fall back into the airway, potentially leading to complications or extended procedure times.

The second working channel may provide further benefits to certain procedures. For example, the second working channel may provide bronchoscopists with the ability to use the main bronchoscope channel for suction while utilizing the additional channel for irrigation. This creates a loop system that significantly improves efficiency and reduces surgical time, addressing one of the most cumbersome and time-consuming aspects of bronchoscopic treatment for pulmonary alveolar proteinosis (PAP).

Bronchial thermoplasty/Rheox procedures can be prolonged due to the vast amount of mucus in the airways of patients with asthma and COPD. The second working channel may provide suction while the main bronchoscope working channel remains occupied by the thermoplasty/Rheox probe. Additionally, oxygen therapy can be provided if needed during the procedure.

The second working channel may help prevent the two components of fibrin glue from combining inside of the working channel of the bronchoscope which may lead to solidification of the glue, rendering the bronchoscope useless. Being able to apply each of the components of the glue inside the airways that provides more precise application, leading to better outcomes for treatment of bronchopleural fistulas or alveolar-pleural fistulas.

The second working channel may allow the bronchoscopist to insufflate the stomach using oxygen while the guidewire is advanced to the stomach via the main bronchoscope working channel. This percutaneous endoscopic gastrostomy (PEG) tube procedure increases the success rate while ensuring adequate apposition of the stomach wall to the abdominal wall, preventing complications.

The second working channel may also improve diagnostic procedures. For example, the second working channel may be utilized for suction while the main working channel operates a cryoprobe, ensuring improved visualization during the diagnostic procedure. Once the biopsy is completed, the second working channel can simultaneously deploy a thermoablative tool or an occlusion balloon to address any bleeding, enhancing patient safety. Additionally, the second working channel may be used to grasp and secure the frozen biopsy tissue with tools such as forceps or a basket, preventing tissue loss during probe thawing and thereby improving the biopsy yield.

In a further implementation, a bronchoscope having a single cylindrical shaped tubular body can also be used in which a first and a second working channel can be used in which both working channels have the same size or they have different sizes to enable use of different tools or therapeutic applications. As shown in FIG. 14, the bronchoscope 400 has a distal end having a distally mounted imaging chip 402, one or more light emitters 404, 406 such as white light emitting diodes (LEDs) a larger working channel 420 and a smaller working channel 410. A catheter 440 having a distal end 450 with imaging aperture or a small diameter tube to view or conduct procedures as previously described herein within the smaller diameter bronchial airways of the patient's lung. An embodiment of this design can include a channel 415 in which a working channel insert can be mounted that slides into position and is connected to the adapter assembly. This embodiment rigidly aligns the two components at the distal end to create a cylindrical tubular body and can enable the use of different size working channels within the working channel inert.

The second working channel may allow use of suction in case of bleeding to maintain a clean field of view. Like the cryobiopsy example, the second working channel may be used to operate a thermoablative tool immediately after the biopsy if bleeding is detected.

It will be appreciated by those skilled in the art that modifications to and variations of the above-described systems and methods may be made without departing from the inventive concepts disclosed herein. Accordingly. the disclosure should not be viewed as limited except as by the scope and spirit of the appended claims.

1. A bronchoscope system comprising:
a bronchoscope having a fiberoptic illumination channel extending through a tubular body, an imaging detector, and a first working channel extending through the tubular body:
a directional actuator to control deflection of a distal tip of the bronchoscope; and
an adapter device attachable to an exterior surface of the tubular body, the adapter device including a second working channel including a port at a proximal end for coupling to an oxygen source.

2. The system of claim 1 wherein the directional actuator positions the distal tip of the bronchoscope in a first direction and a second direction orthogonal to the first direction.

3. The system of claim 1 further comprising a release actuator that separates the adapter device from the bronchoscope.

4. The system of claim 1 wherein the imaging detector is mounted in a proximal section or a distal section of the bronchoscope.

5. The system of claim 1 further comprising a fluid exchange system coupled to at least one bronchoscope channel.

6. The system of claim 1 wherein the tubular body has a diameter in a range of 4 to 8 mm.

7. The system of claim 1 wherein the bronchoscope further comprises a light source.

8. A bronchoscope comprising;
a bronchoscope handle connected to a tubular body for insertion into a bronchial airway of a lung wherein the tubular body has an outer diameter of less than 6 mm and comprises a first working channel, a second working channel, an imaging device and an illumination device, the first working channel having an inner diameter of at least 1.6 mm; and
a port at a proximal end of the second working channel to connect to a fluid source;
a flexible distal tip on a distal end of the tubular body for steering the tubular body through the bronchial tree, the flexible distal tip being operative is response to a directional actuator.

9. The bronchoscope of claim 8 wherein the fluid source comprises an oxygen source.

10. The bronchoscope of claim 8 wherein the second working channel extends through a detachable adapter that extends along the tubular body.

11. The bronchoscope of claim 8 wherein the port is configured to connect to a negative pressure source to apply suction through the second channel.

12. The bronchoscope of claim 8 wherein the first working channel is configured to receive a tool that can be extended through a distal opening of the first working channel.

13. The bronchoscope of claim 12 wherein a further tool is insertable through the second working channel to perform a surgical operation with the bronchial airway.

14. The bronchoscope of claim 12 wherein the tool comprises at least one of a snare, a forceps, a tissue ablation device, a suction tool, a cryoprobe, a retrieval basket, a balloon and a catheter.

15. A method for treating a bronchial condition comprising:
inserting a bronchoscope through the trachea and into a bronchial airway, the bronchoscope have a tubular body, an imaging detector, a light source to illuminate a region within the bronchial airway, a first working channel and a second working channel extending from a proximal end of the tubular body to a distal end;
viewing a portion of the bronchial airway with the bronchoscope;
performing a first operation using the first working channel to treat a condition of the bronchial airway;
during the first operation, performing a therapeutic procedure at a selected location along the bronchial airway using the second working channel.

16. The method of claim 15 wherein a distal end of the bronchoscope has a diameter less than 6 mm.

17. The method of claim 15 wherein the distal end of the bronchoscope is inserted into a depth in at least a third bronchial tree.

18. The method of claim 15 wherein the first operation comprises at least one of a hemoptysis procedure, mucus removal, deployment or removal of a stent, resection of a bronchial tumor, or foreign body removal.

19. The method of claim 15 wherein the bronchoscope comprises a handle attached to the tubular body, an imaging sensor, and a light source.

20. The method of claim 15 further comprising inserting a tool through at least one of the working channels.

21. The method of claim 20 wherein the tool comprises at least one of a snare, a forceps, a tissue ablation device, suction tool, a cryoprobe, a retrieval basket, a balloon, and a catheter.

22. The method of claim 15 further comprising applying suction through at least one of the first working channel and the second working channel.

23. The method of claim 15 wherein the step of inserting the bronchoscope further comprises attaching a working channel adapter, that includes the second working channel, to an outer surface of the bronchoscope.

24. The method of claim 23 wherein a sidewall of the working channel adapter has a concave sidewall having a radius of curvature corresponding to an outer wall of the tubular body.

25. The method claim 23 further comprising coupling a fluid source to a port on the working channel adapter.

26. The method of claim 15 further comprising coupling an oxygen source to at least one of the first working channel and the second working channel.

27. The method of claim 15 further comprising coupling a fluid source to flush a region of a patient's lung through the first working channel and suctioning fluid through the second working channel.
